(19) United States
(12) Patent Application Publication
(10) Pub. No.: US 2018/0315494 A1
(43) Pub. Date: Nov. 1, 2018

(54) REAL-TIME ANTIBIOTIC TREATMENT SUGGESTION

(71) Applicant: KONINKLIJKE PHILIPS N.V., Eindhoven (NL)

(72) Inventors: Raivo Kolde, Melrose, MA (US); Andrew G. Hoss, Cambridge, MA (US); Toon Hendrik Evers, Eindhoven (NL)

(21) Appl. No.: 15/961,959

(22) Filed: Apr. 25, 2018

Related U.S. Application Data
(60) Provisional application No. 62/491,029, filed on Apr. 27, 2017.

(51) Int. Cl. G16H 20/10; G06N 99/00

(52) U.S. Cl. CPC G16H 20/10; G06N 99/005

(57) ABSTRACT

Various embodiments described herein relate to a method, computer readable medium, and device including one or more of the following: receiving at least one newly-available patient feature for the patient; adding the newly-available patient feature to an indication of all available patient features previously established for a previous application of one of a collection of trained models to the patient; comparing the indication of all available patient features to metadata describing input features of respective models of the collection of trained models to determine whether the input features are available for applying the respective trained models to the patient; selecting a selected trained model based on determining that the input features for the selected trained model are available for applying the selected trained model to the patient; and invoking the selected trained model.

### CROSS-REFERENCE To RELATED APPLICATIONS

The present application claims the priority benefit of U.S. provisional application No. 62/491,029 filed Apr. 27, 2017 and entitled “Real-Time Antibiotic Treatment Suggestion,” the entire disclosure of which is hereby incorporated herein by reference for all purposes.

### TECHNICAL FIELD

Various embodiments described herein relate to machine learning and more particularly, but not exclusively, to model selection during rolling acquisition of input data.

### BACKGROUND

In antimicrobial therapy, speed of treatment is one of the most decisive factors for success. Current approaches to studying a pathogen to identify ideal treatment options, however, take a considerable amount of time. While some information, such as gram staining, may be obtained relatively quickly, the complete information provided by antibiotic susceptibility testing (AST) can take 24-48 hours due to the time needed to culture the bacteria.

Due to the staggered and delayed receipt of new information about a particular patient's infection, clinicians often must make a treatment decision based on incomplete information. While evidence-based guidelines for such decisions are provided at the federal, state, and hospital levels, these guidelines are updated relatively infrequently (e.g., about once a year). As such, a clinician is often left unable to adequately react to current outbreaks or changes in antibiotic resistance patterns.

### SUMMARY

Various embodiments described herein relate to a non-transitory machine-readable medium encoded with instructions for execution by a processor for selecting trained model for application to patient data, the non-transitory machine-readable medium comprising: instructions for maintaining an indication of all available patient features for the patient; instructions for receiving newly-available patient features for the patient; instructions for updating the indication of all available patient features to indicate the newly-available patient features; instructions for reading metadata associated with respective trained models of a collection of trained models, wherein the metadata indicates input features of the respective trained models; instructions for comparing the indication of all available patient features to the metadata associated with the respective trained models to determine whether the input features are available for applying the respective trained models to the patient; instructions for selecting a selected trained model based on determining that the input features for the selected trained model are available for applying the selected trained model to the patient; and instructions for invoking the selected trained model.

Various embodiments described herein relate to a method for selecting trained model for application to patient data, the method comprising: receiving at least one newly-available patient feature for the patient; adding the newly-available patient feature to an indication of all available patient features previously established for a previous application of one of a collection of trained models to the patient; comparing the indication of all available patient features to metadata describing input features of respective models of the collection of trained models to determine whether the input features are available for applying the respective trained models to the patient; selecting a selected trained model based on determining that the input features for the selected trained model are available for applying the selected trained model to the patient; and invoking the selected trained model.

Various embodiments described herein relate to a device for selecting trained model for application to patient data, the device comprising a memory and a processor configured to receiving at least one newly-available patient feature for the patient; adding the newly-available patient feature to an indication of all available patient features previously established for a previous application of one of a collection of trained models to the patient; comparing the indication of all available patient features to metadata describing input features of respective models of the collection of trained models to determine whether the input features are available for applying the respective trained models to the patient; selecting a selected trained model based on determining that the input features for the selected trained model are available for applying the selected trained model to the patient; and invoking the selected trained model.

Various embodiments are described wherein the instructions for selecting a selected trained model comprise selecting a trained model having the greatest number of input features among those trained models for which the input features are available for applying the selected trained model to the patient.

Various embodiments are described wherein: the collection of trained models is arranged in a sequence, and the instructions for selecting a selected trained model comprise instructions for selecting a trained model placed furthest in the sequence among those trained models for which the input features are available for applying the selected trained model to the patient.

Various embodiments are described wherein the instructions for invoking the selected trained model comprise instructions for providing the input features to the selected trained model.

Various embodiments are described wherein the instructions for providing the input features to the selected trained model comprise instructions for providing antibiotic sensitivity data for a population of patients to the selected trained model.

Various embodiments additionally include instructions for periodically retraining the respective models of the collection of trained models.

Various embodiments additionally include instructions for generating at least one label after the indication of all available features for the patient includes indications of all features accepted as input by any of the trained models of the collection of models; instructions for generating at least one new training example from the generated at least one label and the available features for the patient; and instructions for adding the at least one new training example to a training set, wherein the instructions for periodically retraining the respective models are configured to train the respective models using the training set.

Various embodiments are described wherein periodically retraining the respective models of the collection of trained models comprises training the respective models using training examples derived from antibiotic sensitivity data for a population of patients.

Various embodiments are described wherein a first model of the collection of trained models is configured to accept as an input feature first data obtained from a first procedure that takes a first amount of time; and a second model of the collection of trained models is configured to accept as an input feature second data obtained from a second procedure that takes a second amount of time that is longer than the first amount of time.

Various embodiments are described a first model of the collection of trained models is configured to accept at least one early genetic sequencing value comprising at least one of pathogen species and pathogen genes associated with antibiotic resistance; and a second model of the collection of trained models is configured to accept the at least one early genetic sequencing value and at least one late genetic sequencing value comprising at least one of a pathogen multilocus sequence type, pathogen single nucleotide polymorphisms associated with antibiotic resistance, pathogen single nucleotide polymorphisms associated with a core genome, and pathogen genes labeled as ancillary, wherein the first model does not accept the at least one late genetic sequencing value as input.

### BRIEF DESCRIPTION OF THE DRAWINGS

In order to better understand various example embodiments, reference is made to the accompanying drawings, wherein:

FIG. 1 illustrates an example timeline of patient treatment using an embodiment of the present system;

FIG. 2 illustrates an example functional diagram of an embodiment of the present system;

FIG. 3 illustrates an example illustration of the operation of a model selector according to various embodiments;

FIG. 4 illustrates an example hardware device for implementing various embodiments;

FIG. 5 illustrates example metadata for use in the operation of various embodiments;

FIG. 6 illustrates an example method for extracting and curating features available for a patient;

FIG. 7 illustrates an example method for selecting a model for application to available patient features; and

FIG. 8 illustrates an example display for presenting model outputs to a user.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

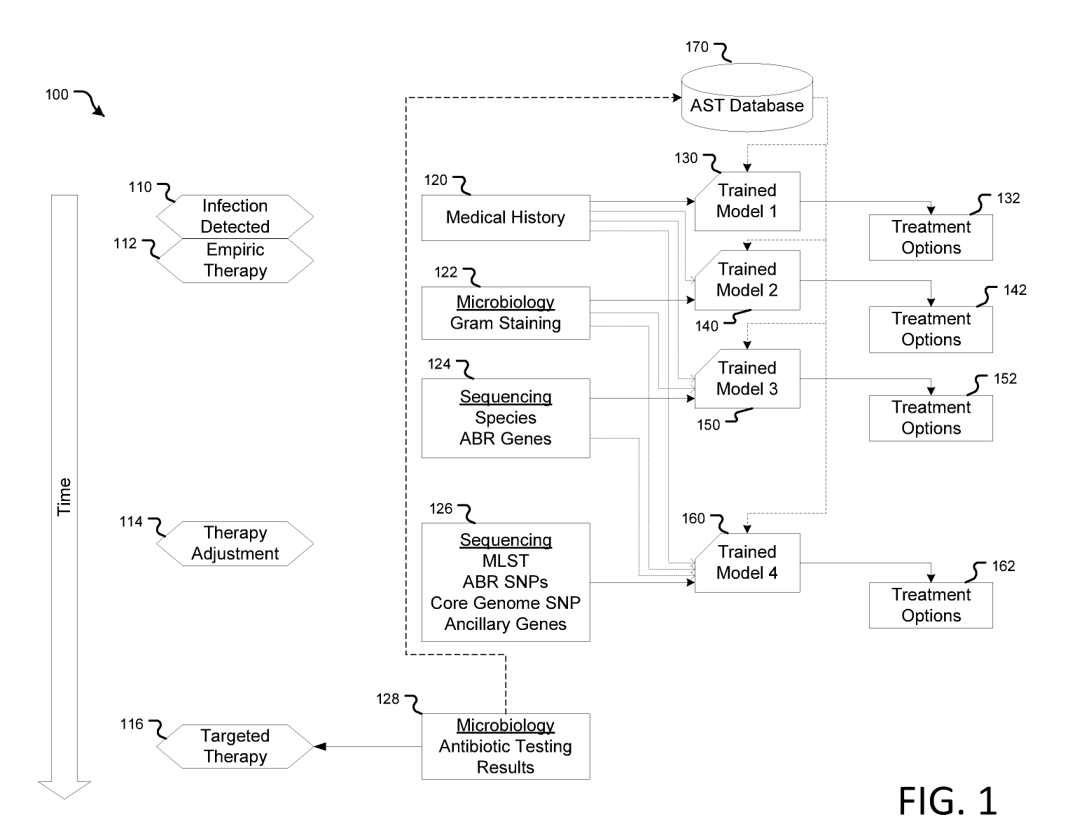

FIG. 1 illustrates an example timeline 100 of patient treatment using an embodiment of the present system. According to some typical treatment workflows, treatment of patient begins at the time that an infection is detected 110 by beginning empiric therapy 112, that is, therapy selected based on the best information available about the patient's condition. For example, the clinician may choose a treatment based on information such as patient medical history 120, clinical observations, the current hospital-wide antibiogram, etc. At this point in time, specific information about the infection itself is unavailable. As time goes on, additional infection- or pathogen-specific information may be acquired. For example, microbiology information 122, 128 such as gram staining results and antibiotic testing results; or genetic sequencing information 124, 126 such as pathogen species, antibiotic resistance (ABR)-associated genes, multi-locus sequence typing (MLST), ABR-related single nucleotide polymorphisms (SNP), core genome SNPs, and ancillary genes. As shown, such information may be made available at different points in time; for example, gram staining results may be available before a determination of pathogen species from genetic sequencing, which may be available before full antibiotic testing results are available. While the full antibiotic testing results, once available, may indicate a specific targeted therapy 116 for the patient, the clinician may also, at some point before the antibiotic testing result are available, use the additional information available in the interim to adjust 114 the originally prescribed therapy 112 one or more times.

Various embodiments present a system that operates within this workflow. For example, various embodiments include a collection of one or more trained models 130, 140, 150, 160, each configured to receive some of the available information about a patient and generate one or more treatment options 132, 142, 152, 162 (e.g., one or more antibiotics that are suggested for administration to the patient). The trained models 130, 140, 150, 160 may be virtually any machine learning model useful for selecting treatment options such as, for example, regression models, neural networks, deep learning architectures, or groups of any of these or other models. For example, a trained model may include multiple classifiers, one trained for making recommendations with regard to each possible antibiotic; accordingly, as used herein the terms “trained model” and “model” will be understood to additionally include groupings of multiple trained models that are to be applied to patient data together. According to various embodiments, each model 130, 140, 150, 160 may be trained to accept different subsets of possible patient inputs and generate treatment options 132, 142, 152, 162 therefrom. As such, different ones of the models 130, 140, 150, 160 may be applicable at different times in the patient's treatment, depending on the available information. For example, if the patient's medical history and the pathogen species are example, one model may be applicable, while if the patient's medical history and gram staining results are available, a different model may be applicable. In some cases, inputs may be available for more than one of the models, in which case the system may select one model from the multiple candidates for application to the presently available inputs. In some embodiments, the models 130, 140, 150, 160 may be arranged in a sequence such that each model in the sequence accepts additional information beyond the inputs accepted for the previous model. For example, as shown in the illustrated system of FIG. 1, trained model 1 130 may accept the patient's medical history, while trained model 2 140 may accept the both the patient's medical history 120 and gram staining results 122. In some such embodiments, the system may opt to apply the model that incorporates the greatest number of available inputs; so, the trained model 1 130 may be applied until such time as the gram staining results 122 are available, at which point the system may switch to applying trained model 2 140. Various alternative sequences of models (e.g., non-linear or tree sequences) and criteria for selection among multiple possibly-applicable models will be apparent in view of the following description.

According to various embodiments, models 130, 140, 150, 160 are applied to available information to produce recommended treatment options 132, 142, 152, 162 until such time that full antibiotic testing results 128 are available for defining a targeted therapy 116. At that time, the antibiotic testing results 128 may then be stored in an antibiotic sensitivity testing (AST) database 170. This AST database 170 may be maintained for all patients in a hospital, in a hospital system, in a geographic location, across the country, or across the world. Thereafter, the AST database 170 may be used to periodically retrain the models 130, 140, 150, 160 to account for changes in the bacterial ecosystem to which the AST database 170 corresponds. Thus, for example, if recent antibiotic sensitivity testing has revealed a sharp increase in resistance to amoxicillin in the present hospital, the models 130, 140, 150, 160 may be retrained to take this into account and (likely) tend to suggest other treatment options.

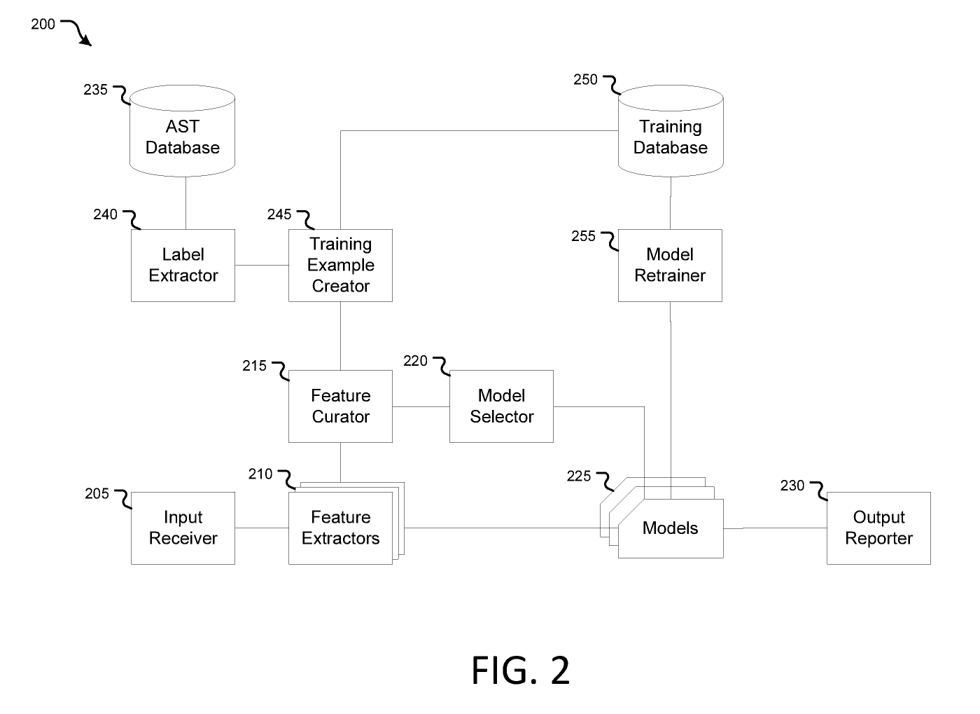

FIG. 2 illustrates an example functional diagram 200 of an embodiment of the present system. The functional diagram 200 illustrates numerous functional blocks that may correspond to one or more hardware devices. For example, in some embodiments, a single hardware device may implement all functions and storage depicted in the diagram 200 while, in other embodiments, a each function and storage may correspond to a separate, dedicated hardware device. Virtually any arrangement therebetween may also be implemented. For example, one on-premise device (or several instances thereof) may implement functions 205-230 to provide treatment suggestions during treatment, a cloud storage device may maintain the AST database 235, and a cloud virtual machine may implement functions/storage 240-260 to periodically retrain the models 225 and push the newly-trained versions to the on-premise device. Various additional arrangements will be apparent for implementing the teachings and principles described herein.

Initially, an input receiver 205 may receive information about a particular patient such as medical history, demographic information, microbiology lab results, genetic sequencing results, or any other information that may be pertinent to selecting a therapy. The information may be received by the input receiver 205 actively pulling the information from another device (e.g., an EMR system) or passively receiving information pushed from another device. In various embodiments, the input receiver 205 may be configured to handle information in multiple different forms, such as structured reports, free-text, images, discrete values (e.g., a packet carrying one or more attribute-value pairs), and other forms.

Upon receiving new information, the input receiver 205 may pass the new information to one or more feature extractors 210 for processing the information into a form suitable for use as model input. For example, where the input is a structured report, a feature extractor may be employed to select and extract only those values deemed relevant by the system. As another example, for a free text report, a feature extractor 210 may be employed to perform natural language processing on the report to locate pertinent information. Thus, the input receiver 205 may choose one or more feature extractors 210 from the available group based on the input format or its source.

As noted above, before the input is provided to one of the models 225, the system 200 chooses which model 225 of those available is to be applied. Specifically, in various embodiments, the system selects a model 225 based on what model inputs are available for the patient, whether just received or previously received on a previous application of one of the models 225. To support model selection, a feature curator maintains at least an indication of which model inputs are available for the patient. For example, upon receiving any new features from a feature extractor 210, the feature curator may update a table associated with the patient to indicate the newly received feature or may store the actual feature value for the patient. Alternatively, the feature curator 215 may, rather than keeping a running indication of available features, be adapted to gather the features or the knowledge of which features are available at the time of each model application by, for example, requesting information be delivered from another system (e.g., an EMR system) from which the features may be extracted anew.

Once the feature curator 215 has an up-to-date indication of which features are available for model application, a model selector 220 may select a model (or, in some embodiments, multiple models) for application to the patient's features. Various methods for judging which model is to be chosen may be employed. For example, in some embodiments, the model selector 220 may choose, from those models where all features are available, whichever model accepts the greatest number of inputs. As another example, each model may be associated with a weight, and the model selector may choose, from those models where all features are available, whichever model has the greatest weight. In some embodiments, the model selector 220 may itself be a model (e.g., a neural network) trained to select which model 225 is to be applied based on retrospective data of previous model choices and labels derived from, for example, whether the clinician accepted the recommendation. In some embodiments, one or more of the models 225 may be capable of handling at least some missing inputs (e.g., using feature imputation techniques). In such embodiments, the model selector 220 may use other methods to determine which model 225 is to be applied. For example, each model may be associated with metadata indicating which features are required and which are optional; in some such embodiments, the model selector may choose from those models for which all required features are available. In other embodiments, each model may be associated with metadata indicating an “importance” score for each feature it accepts as input; in such embodiments, the model selector 220 may tally a score for each model based on the available features and their per-model importance score, then select the model with the highest score.

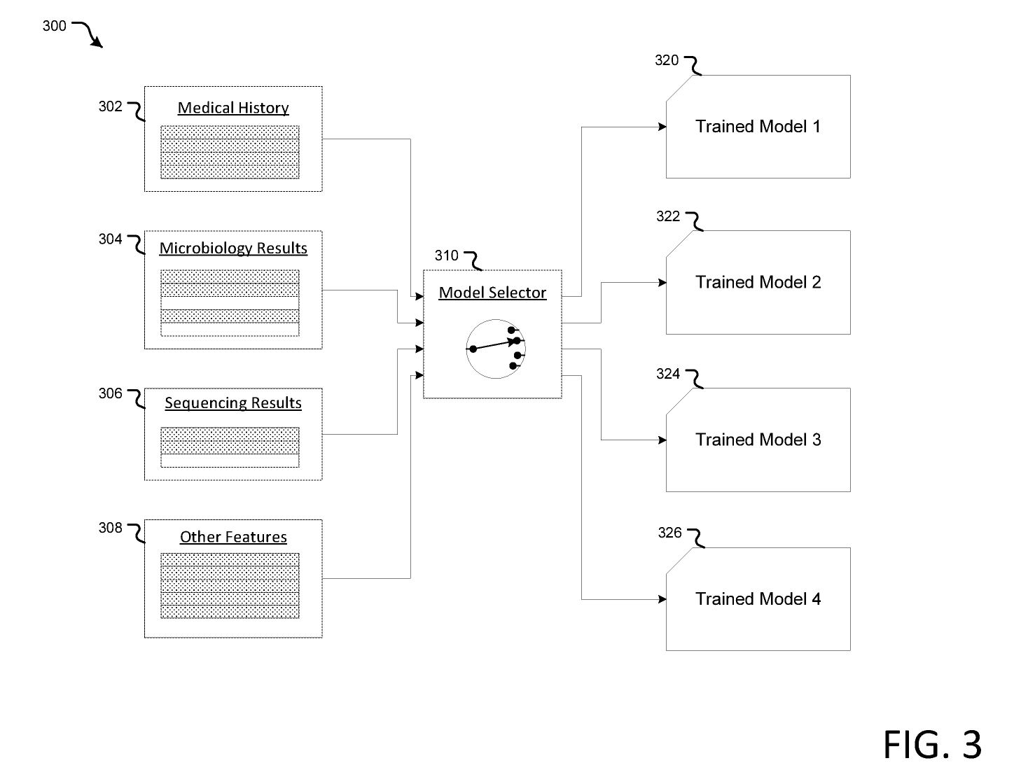

FIG. 3 illustrates an example illustration 300 of the operation of a model selector according to various embodiments such as, for example, model selector 220 of FIG. 2. As noted, a feature curator may maintain one or more data structures 302, 304, 306, 308 that indicates, of all the features used by at least one model 320, 322, 324, 326, which are available for the current patient. The data structures 302, 304, 306, 308 may be separate data structures or a single data structure. The term “feature matrix” as used herein will be understood to refer to any data structure (to include matrices, arrays, trees, bitstrings, etc.) for maintaining at least an indication of available features for one or more patients. For example, the data structures may store a Boolean value for each feature or the feature value itself. As shown in the illustrated example, all possible medical history 302 values are available while only three of five microbiology results 304 values are available (as shown by grey and white boxes). Based on the indication of which features are available and which are not, the model selector 310 chooses one of the models 320, 322, 324, 326 for application to the available features; as shown, the model selector 310 has currently chosen model 2 322, which will be used to generate therapy recommendations. At some point in the future, the system may receive additional sequencing results 306 such that the currently unfilled box is filled, to indicate the presence of an additional feature for the patient. At that point, the model selector 210 may select model 3 324 for application to the patient's features and generate new, updated recommendations.

Returning to FIG. 2, once the model selector 220 has selected a model 225 for application, the patient features are provided to that model 225 by the feature extractors or, in some embodiments, the feature curator. The models 225 then produce an output comprising a therapy recommendation for the patient, which is then presented to a clinician via the output reporter. In various embodiments, the models 225 may output one or more “raw” scores or other values that are translated into human readable form by the output reporter 230. For example, many classifiers that may be used to implement the models 225 may include a final stage Sigmoid function and, as such, outputs may simply be one or more floating point numbers between 0 and 1. The output reporter 230 may then interpret the model output by correlating it to one or more therapies to be delivered. For example, where the model is associated with amoxicillin, the output reporter 230 may compare the floating point number to a threshold to determine whether or not amoxicillin is to be recommended. Where the model outputs multiple such numbers, the output reporter 230 may compare the floating point numbers to one or more thresholds to determine which one or more therapies are to be recommended. In some embodiments, the output reporter 230 may also derive a confidence in a recommendation for presentation to the user. In some embodiments, the output reporter 230 may also generate an interface (e.g., an HTML page) for presentation to the user including the recommendation(s) and other information such as recommendation confidence, information about the recommendations (e.g., as retrieved from an external source), information about the patient such as demographic information or features which had an impact on the ultimate recommendation, or other information.

In some embodiments, the system may be adapted to provide therapy directly, in addition to or instead of making recommendations to the clinician. For example, where a recommendation is administration of a medication, the output reporter 230 may enter a pharmacy order via an API to an order entry system or control an infusion pump attached to the patient to deliver the medication. As another example, where a recommended therapy is a change to a ventilator setting (e.g., where the principles taught herein are applied to conditions other than pathogenic infection), the output reporter may configure the ventilator via an interface thereto. Various other actions for the output reporter will be apparent.

As noted above, during the course of treatment of a patient, at some point full antibiotic sensitivity testing results will be made available and stored in one or more AST databases. For example, the AST database(s) may be a hospital database or a nationwide database that stores the results of antibiotic sensitivity testing correlated to a patient or otherwise associable by the system 200 with the features collected for a particular patient. As new results are made available in the AST database, a label extractor 240 may determine, for a previously-treated patient, which therapies were truly the best choices. For example, the label extractor may take note of which antibiotics the infection was deemed susceptible and resistant for use in future retraining of the models 225. A training example creator 245 may then create one or more training examples from the newly extracted label and the features acquired for the patient. For example, the training example creator 245 may create a record including all features held by the feature curator 215 for the patient, and a “true” or “false” label for each antibiotic indicating whether the AST results reveal susceptibility or resistance. The training example creator 245 may then store the training example in a training database 250 for future use.

Periodically, the model retrainer 255 may execute a machine learning algorithm to retrain the models 225 based on new training examples stored in the training database 250 since the last time them models 225 were retrained. For example, the model retrainer 255 may execute every hour, day, week, or on some other timed basis. Alternatively, the model retrainer 255 may execute whenever a predetermined number of new training examples (e.g., 100, 1000, etc.) have been added to the training database 250. Any appropriate training algorithm may be used for the chosen models 225. For example, gradient descent may be used for regression models or back propagation may be used for neural networks. In some embodiments, the model retrainer 255 may retrain the models 225 based on all training examples stored in the training database, new or old. In some embodiments, the model retrainer 255 may first delete old training examples, such as those examples older than a predetermined age (e.g., 7 days, 3 months, etc.). In some embodiments, the model retrainer 255 may place heavier emphasis on examples that are newer than those that are older. Using these or other similar methods, the model retrainer 255 periodically attempts to update the models 225 to take into account the current pathogenic ecosystem as reflected by the AST database 235.

According to various alternative embodiments, rather than retraining the models 225 to “bake in” the current antibiotic sensitivity trends to the models 225, the models may instead be trained to accept one or more indicators of antibiotic sensitivity trends as input. For example, a collection of features representing the hospital's current antibiogram may be included as inputs for each model. In some such embodiments, the model retrainer 255 may still be employed to periodically retrain the models simply to take advantage of the additional training examples generated by the system 200.

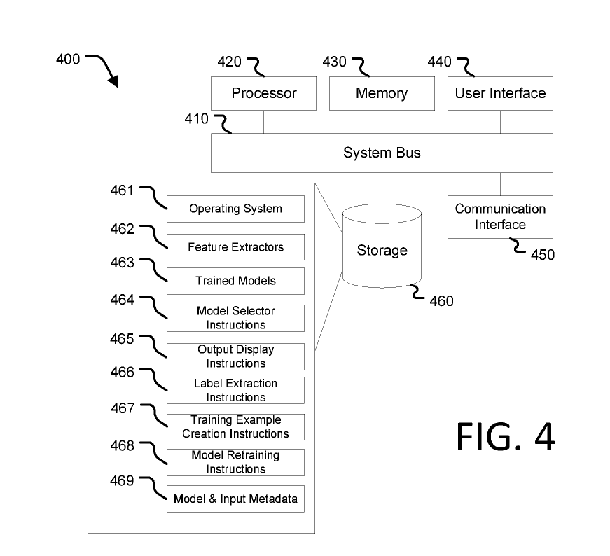

FIG. 4 illustrates an example hardware device 400 for implementing various embodiments. The exemplary hardware 400 may correspond to one or more of the functional blocks of the system 200 described in FIG. 2. As shown, the device 400 includes a processor 420, memory 430, user interface 440, communication interface 450, and storage 460 interconnected via one or more system buses 410. It will be understood that FIG. 4 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, field programmable gate array (FPGA), application-specific integrated circuit (ASIC), or other similar devices.

The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. It will be apparent that, in embodiments where the processor includes one or more ASICs (or other processing devices) that implement one or more of the functions described herein in hardware, the software described as corresponding to such functionality in other embodiments may be omitted.

The user interface 440 may include one or more devices for enabling communication with a user such as an administrator. For example, the user interface 440 may include a display, a mouse, and a keyboard for receiving user commands. In some embodiments, the user interface 440 may include a command line interface or graphical user interface that may be presented to a remote terminal via the communication interface 450.

The communication interface 450 may include one or more devices for enabling communication with other hardware devices. For example, the communication interface 450 may include a network interface card (NIC) configured to communicate according to the Ethernet protocol. Additionally, the communication interface 450 may implement a TCP/IP stack for communication according to the TCP/IP protocols. Various alternative or additional hardware or configurations for the communication interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon with the processor 420 may operate. For example, the storage 460 may store a base operating system 461 for controlling various basic operations of the hardware 400.

The storage 460 may also include instructions defining one or more feature extractors 462 for placing incoming patient information in form for consumption by one or more trained models 463. The trained models 463, in turn, may comprise instructions (e.g., model architecture and learned weights) for consuming features and outputting one or more therapy recommendations. Model selector instructions 464 may be provided for determining, based on those features that are available (e.g., as may be indicated by feature curator instructions, not shown), which model 464 should be applied. To support this decision, the storage 460 may include metadata 469 identifying the types of features that may be used and, for each model, which features are consumed as input. Output display instructions 465 may include instructions for interpreting raw output from models and presenting such interpretations in a human readable form (e.g., a formatted webpage or report).

For retraining the models 463, the storage 460 may include label extraction instructions 466 for interfacing (e.g., via the communications interface 450) with one or more AST databases to determine the true resistance profile of a patient's infection and create one or more labels for use in constructing a training example. Training example creation instructions 467 may then compile these labels together with features available for the patient to create one or more records to be used as training examples. Such training example may be stored in a local or remote training database (not shown). Model retraining instructions 468 may then include instructions for periodically retraining the models 463 based on new training examples, potentially deleting or deemphasizing older training examples so as to account for recent hospital or geographic trends in resistance profiles.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered to be “non-transitory machine-readable media.” As used herein, the term “non-transitory” will be understood to exclude transitory signals but to include all forms of storage, including both volatile and non-volatile memories.

While the host device 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Further, where the device 400 is implemented in a cloud computing system, the various hardware components may belong to separate physical systems. For example, the processor 420 may include a first processor in a first server and a second processor in a second server.

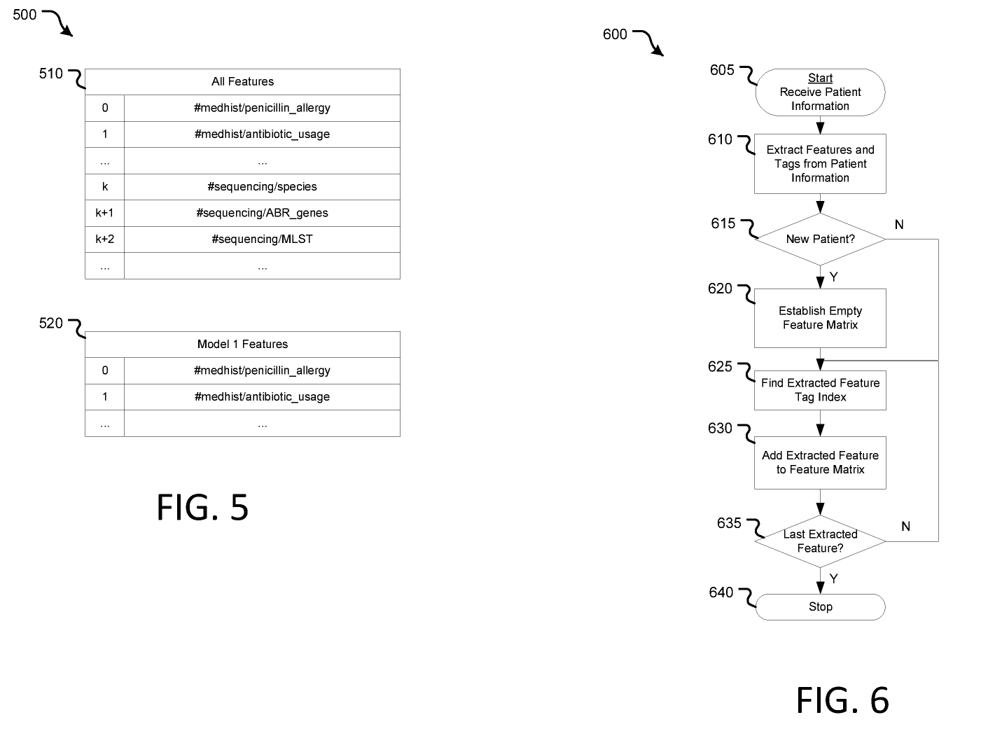

FIG. 5 illustrates example metadata 500 for use in the operation of various embodiments. The metadata may correspond, for example, to the metadata 469 of FIG. 4 and may be used to determine which features are consumed by each model for the purposes of model selection.

An all features metadata table 510 may indicate each feature known to the system. For example, the all features metadata table 510 may be constructed as the union of all features indicated by any model as an input. Alternatively, each feature extractor or external data source may register one or more tags with the system to indicate which features will be provided thereby; such registered tags may then be added to the all features metadata table 510. Such registration functionality may be leveraged to make the system flexible and extendible, whereby feature extractors or external data sources may be added or updated registered on-the-fly.

As shown, each feature may be described by way of a tag. For example, “#medhist/penicillin_allergy” may be a tag corresponding to a feature specifying whether the patient has a penicillin allergy. Similarly, a “#sequencing/species” tag may be a tag corresponding to a feature specifying the pathogen species as determined by genetic sequencing. Such tags are shown organized in a hierarchical arrangement, though other arrangements are possible. In some embodiments, the tags may not be human readable. For example, tags may simply be unique identifiers randomly assigned at the time of input or model registration.

Similarly, a model 1 metadata table 520 may indicate the feature tags associated with the features consumed by model 1 as input. Such table may be populated, for example, at the time model 1 is introduced to the system. For example, model 1 may register itself with the system by advertising tags needed for generating a therapy recommendation. Such registration functionality may be leveraged to make the system flexible and extendible, whereby models may be added or updated registered on-the-fly. Similar metadata tables (not shown) may be maintained for each model registered with the system.

FIG. 6 illustrates an example method 600 for extracting and curating features available for a patient. The method 600 may be performed, for example, by the feature extractor instructions 462 and other instructions of FIG. 4 (such as input receiver and feature curator instructions, not shown) to support selection of a model by the system. Various alternative methods for extracting and curating features available for a patient will be apparent.

The method 600 begins in step 605 where the device receives new patient information such as, for example, a structured report, free text, image data, or individual values. In step 610, the device extracts any features from the new patient information along with metadata tags. For example, the patient information may include the metadata tags already upon receipt or individual feature extractors may attach the tags at the time of feature extraction. As explained above, such metadata tags may be used to keep track of which features are available and which features are needed for each model.

In step 615, the device determines whether the new patient information relates to a new (to the system) patient. For example, the device may read a patient identifier from the received patient information and determine whether any features are already locally-stored in association with that patient identifier. If the patient is new, the device establishes an empty feature matrix for the new patient. For example, the device may establish a new bitstring, initially all zeroes, including one bit for each feature known to the system (e.g., by determining the length of the all features metadata table 510). Alternatively, the device may establish a new array, initially all empty values, including one position for each feature known to the system. Next, in step 625, the device finds the index for the extracted feature tag and, in step 630, adds the extracted feature to the feature matrix. For example, where the feature matrix is a bitstring, the device may locate the tag in the all features metadata table 510 and retrieve the corresponding numerical index. In the illustrated example, if the tag is “#medhist/antibiotic_usage,” the index may be “1.” The system may then set the bit at the located index within the bitstring to a value of “1” to indicate the feature is present. As another example, where the feature matrix is an array, the system may store the feature value at the located index within the array.

It will be appreciated that alternative indexing strategies may be used rather than a tag-to-index lookup. For example, a dictionary data structure may be used where the tag itself serves as a key into the data structure. Alternatively, the system-wide tag may be the index itself (e.g., the tags may be “0,” “1,” “2,” etc.).

In step 635 the device determines whether there are any features that were extracted in step 610 that remain to be added to the matrix. If so, the method loops back to step 625. Once the last extracted features has been processed, the method proceeds to end in step 635. In various embodiments, before ending, the method 600 may invoke, flow into, or otherwise begin the next algorithm in the system, such as the model selector instructions 464.

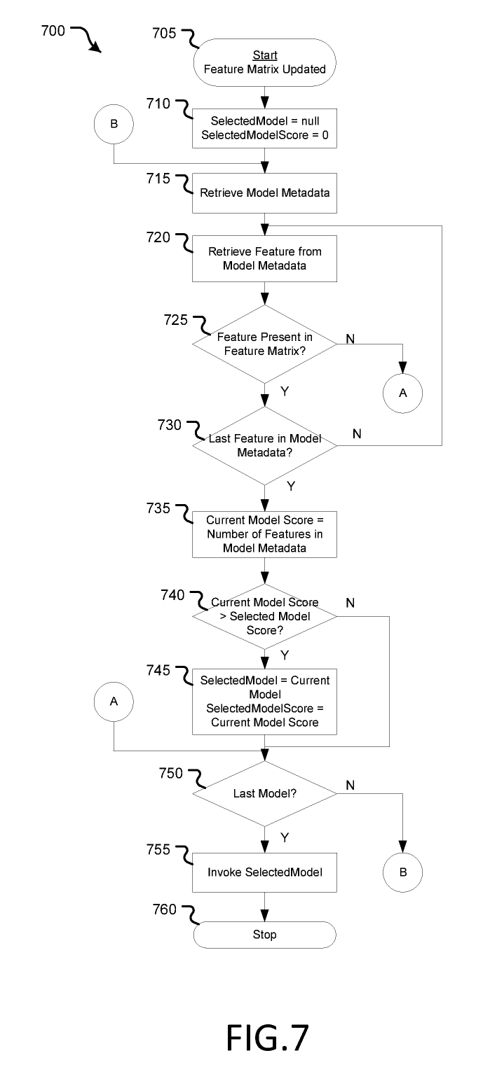

FIG. 7 illustrates an example method 700 for selecting a model for application to available patient features. The method 700 may be performed, for example, by the model selector instructions 464. Various alternative methods for selecting a model will be apparent.

FIG. 7 begins in step 705 once the feature matrix has been updated, either with a newly available feature or, in some embodiments, a new value for a previously-available feature. In step 710, the device initializes two working variables, SelectedModel and SelectedModelScore, to keep track of the highest “scoring” model while iterating through all of the system's models, where the model with the highest score will be selected according to the present example algorithm. In step 715, the device retrieves metadata for a model to be analyzed on this iteration. For example, the device may retrieve the model metadata table 520 of FIG. 5.

The device then begins to iterate through the table for the present model by retrieving a feature listed in the model metadata in step 720. In step 725, the device determines whether the current feature for the current model is listed as available in the feature matrix. If not, then the device may determine that the needed inputs are not available to apply the current model, and as such the method should skip to the next model (if any) by skipping ahead to step 750. Otherwise, if the feature is indicated as available in the feature matrix, the method 700 proceeds to step 730 where the device determines whether additional features remain in the model metadata for the current model. If the current feature is not the last to be considered, the method loops back to step 720 to continue its consideration of all input features for the current model. Once all features have been considered and deemed present by the feature matrix, the method proceeds to step 735.

In step 735, the device, having the determined that the model can be applied begins to determine whether the model is the “best” model to apply according to some metric. According to this example, the model that consumes the greatest number of inputs is deemed the “best” though various alternative embodiments may employ different approaches to selecting one (or more) model from multiple potentially applicable models. Following the illustrated example, the device computes a current model score by counting the number of features accepted by the model as input (e.g., the length of the model metadata table). In step 740, the device determines whether the current model score exceeds the score currently stored in the SelectedModelScore variable. If so, the method proceeds to step 745 where the SelectedModelScore is updated to store the current model score, and the SelectedModel variable is updated to store an identifier of the current model. If, on the other hand, the current score does not exceed the SelectedModelScore variable in step 740, the method 700 skips ahead to step 750, leaving the working variables unchanged thereby maintaining the previous tentative selection of a model.

The method 700 proceeds to iterate through all of the system's models by looping back to step 715 until the last model is processed. The method 700 then proceeds to step 755, where the device invokes whichever model is now identified by the SelectedModel variable. That model may then consume the available features and then produce an output for presentation by the system to a user. The method 700 then proceeds to end in step 760.

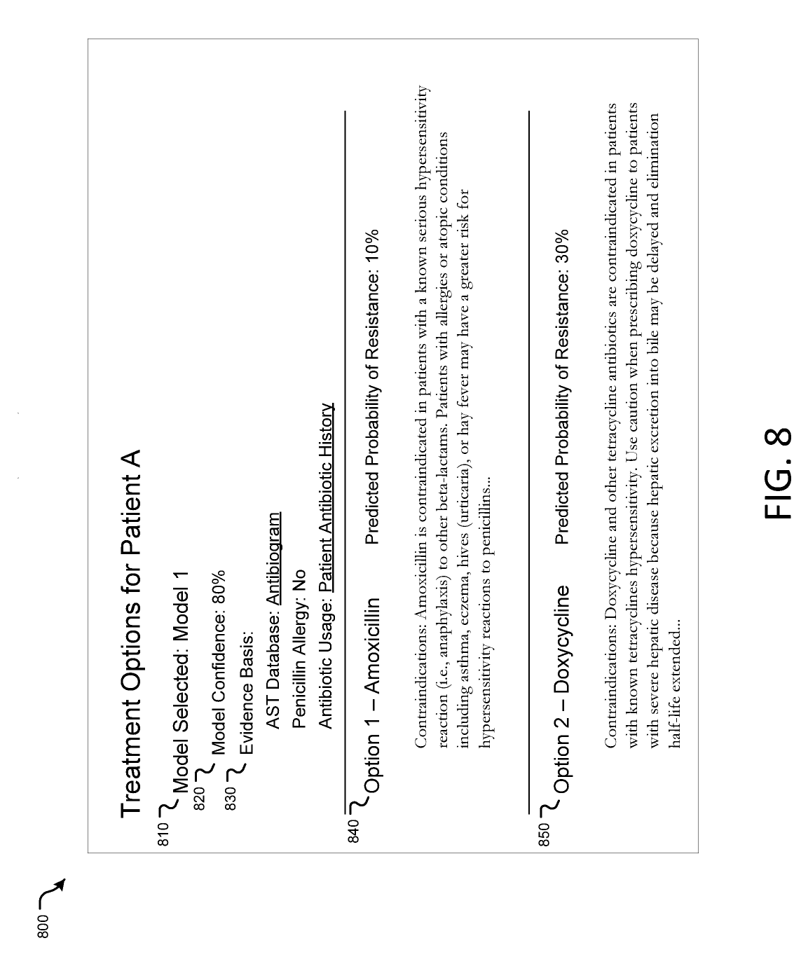

FIG. 8 illustrates an example display 800 for presenting model outputs to a user. As noted above, an output reporter may interpret model output and generate a human-readable version of the output, such as a web page or report document. As shown, one example of an output 800 may include information about how the recommendations were generated such as an indication of which model was applied 810, a confidence in the model based on the supplied inputs 820, and an evidence basis indicating which features most heavily weighed in favor of the presented treatment options. Various methods for interpreting the operation of a model an relative feature importance will be apparent.

The display 800 may include one or more recommended therapies along with additional information such as a predicted probability of drug resistance (where models provide such information) and a description of the treatment (e.g., as retrieved from an external knowledgebase or other source). The display 800 lists two such treatments 840, 850 to provide the clinician a selection of possible options for treating the patient. To generate such alternative recommendations, as noted above, some models may include a separate classifier for each possible treatment. Those classifiers providing outputs above a respective threshold may then be listed on the display 800, e.g., in the order of confidence, predicted probability of resistance, or other criteria.

According to the foregoing, various embodiments provide a system for generating machine learning output based on rolling acquisition of input data. Particularly, by training multiple models on different subsets of the full feature set, an appropriate model can be selected for application at any time. Such functionality may be particularly beneficial in situations where model output is desired or even needed before all input features are available, such as in the case of acute care of patients and infection control. Further, by periodically retraining such models based on changing environment dynamics, such as resistance profile trends reflected in a database of completed antibiotic sensitivity tests, clinical recommendations can take into account recent data that typically is not available to clinicians.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A non-transitory machine-readable medium encoded with instructions for execution by a processor for selecting trained model for application to patient data, the non-transitory machine-readable medium comprising:
instructions for maintaining an indication of all available patient features for the patient;
instructions for receiving newly-available patient features for the patient;
instructions for updating the indication of all available patient features to indicate the newly-available patient features;
instructions for reading metadata associated with respective trained models of a collection of trained models, wherein the metadata indicates input features of the respective trained models;
instructions for comparing the indication of all available patient features to the metadata associated with the respective trained models to determine whether the input features are available for applying the respective trained models to the patient;
instructions for selecting a selected trained model based on determining that the input features for the selected trained model are available for applying the selected trained model to the patient; and
instructions for invoking the selected trained model.

2. The non-transitory machine-readable medium of claim 1, wherein the instructions for selecting a selected trained model comprise selecting a trained model having the greatest number of input features among those trained models for which the input features are available for applying the selected trained model to the patient.

3. The non-transitory machine-readable medium of claim 1, wherein:
the collection of trained models is arranged in a sequence, and
the instructions for selecting a selected trained model comprise instructions for selecting a trained model placed furthest in the sequence among those trained models for which the input features are available for applying the selected trained model to the patient.

4. The non-transitory machine-readable medium of claim 1, wherein the instructions for invoking the selected trained model comprise instructions for providing the input features to the selected trained model.

5. The non-transitory machine-readable medium of claim 4, wherein the instructions for providing the input features to the selected trained model comprise instructions for providing antibiotic sensitivity data for a population of patients to the selected trained model.

6. The non-transitory machine-readable medium of claim 1, further comprising instructions for periodically retraining the respective models of the collection of trained models.

7. The non-transitory machine-readable medium of claim 6, further comprising:
instructions for generating at least one label after the indication of all available features for the patient includes indications of all features accepted as input by any of the trained models of the collection of models;
instructions for generating at least one new training example from the generated at least one label and the available features for the patient; and
instructions for adding the at least one new training example to a training set,
wherein the instructions for periodically retraining the respective models are configured to train the respective models using the training set.

8. The non-transitory machine-readable medium of claim 6, wherein periodically retraining the respective models of the collection of trained models comprises training the respective models using training examples derived from antibiotic sensitivity data for a population of patients.

9. The non-transitory machine-readable medium of claim 1, further comprising the collection of models, wherein:
a first model of the collection of trained models is configured to accept as an input feature first data obtained from a first procedure that takes a first amount of time; and
a second model of the collection of trained models is configured to accept as an input feature second data obtained from a second procedure that takes a second amount of time that is longer than the first amount of time.

10. The non-transitory machine-readable medium of claim 1, further comprising the collection of models, wherein:
a first model of the collection of trained models is configured to accept at least one early genetic sequencing value comprising at least one of pathogen species and pathogen genes associated with antibiotic resistance; and
a second model of the collection of trained models is configured to accept the at least one early genetic sequencing value and at least one late genetic sequencing value comprising at least one of a pathogen multilocus sequence type, pathogen single nucleotide polymorphisms associated with antibiotic resistance, pathogen single nucleotide polymorphisms associated with a core genome, and pathogen genes labeled as ancillary,
wherein the first model does not accept the at least one late genetic sequencing value as input.

11. A method for selecting trained model for application to patient data, the method comprising:
receiving at least one newly-available patient feature for the patient;
adding the newly-available patient feature to an indication of all available patient features previously established for a previous application of one of a collection of trained models to the patient;
comparing the indication of all available patient features to metadata describing input features of respective models of the collection of trained models to determine whether the input features are available for applying the respective trained models to the patient;
selecting a selected trained model based on determining that the input features for the selected trained model are available for applying the selected trained model to the patient; and
invoking the selected trained model.

12. The method of claim 11, wherein selecting a selected trained model comprises selecting a trained model having the greatest number of input features among those trained models for which the input features are available for applying the selected trained model to the patient.

13. The method of claim 11, wherein:
the collection of trained models is arranged in a sequence, and
selecting a selected trained model comprises selecting a trained model placed furthest in the sequence among those trained models for which the input features are available for applying the selected trained model to the patient.

14. The method of claim 11, wherein invoking the selected trained model comprises providing the input features to the selected trained model.

15. The method of claim 14, wherein providing the input features to the selected trained model comprises providing antibiotic sensitivity data for a population of patients to the selected trained model.

16. The method of claim 11, further comprising periodically retraining the respective models of the collection of trained models.

17. The method of claim 16, further comprising:
generating at least one label after the indication of all available features for the patient includes indications of all features accepted as input by any of the trained models of the collection of models;
generating at least one new training example from the generated at least one label and the available features for the patient; and
adding the at least one new training example to a training set,
wherein the step of periodically retraining the respective models comprises training the respective models using the training set.

18. The method of claim 16 wherein periodically retraining the respective models of the collection of trained models comprises training the respective models using training examples derived from antibiotic sensitivity data for a population of patients.

19. The method of claim 11, wherein:
a first model of the collection of trained models is configured to accept as an input feature first data obtained from a first procedure that takes a first amount of time; and
a second model of the collection of trained models is configured to accept as an input feature second data obtained from a second procedure that takes a second amount of time that is longer than the first amount of time.

20. The method of claim 11, wherein:
a first model of the collection of trained models is configured to accept at least one early genetic sequencing value comprising at least one of pathogen species and pathogen genes associated with antibiotic resistance; and
a second model of the collection of trained models is configured to accept the at least one early genetic sequencing value and at least one late genetic sequencing value comprising at least one of a pathogen multilocus sequence type, pathogen single nucleotide polymorphisms associated with antibiotic resistance, pathogen single nucleotide polymorphisms associated with a core genome, and pathogen genes labeled as ancillary,
wherein the first model does not accept the at least one late genetic sequencing value as input.
